(19) United States
(12) Patent Application Publication
(10) Pub. No.: US 2026/0108174 A1
(43) Pub. Date: Apr. 23, 2026

(54) OPERATION OF A MEDICAL DEVICE SYSTEM TO IDENTIFY A FALL EVENT AS A CARDIAC FALL EVENT

(71) Applicant: Medtronic, Inc., Minneapolis, MN (US)

(72) Inventors: Gautham Rajagopal, Minneapolis, MN (US); Yong K. Cho, Excelsior, MN (US)

(21) Appl. No.: 19/179,465

(22) Filed: Apr. 15, 2025

Related U.S. Application Data
(60) Provisional application No. 63/635,101, filed on Apr. 17, 2024.

(51) Int. Cl. A61B 5/11; A61B 5/0205; A61B 5/287; A61B 5/349

(52) U.S. Cl. CPC A61B 5/1117; A61B 5/0205; A61B 5/287; A61B 5/349; A61B 2562/0219

(57) ABSTRACT

An example system includes an implantable medical device includes determine the patient suffered a fall event; in response to the determination that the patient suffered the fall event, determine whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identify the fall event as a cardiac fall event.

### RELATED APPLICATIONS

This application claims the benefit of U.S. Provisional Application Ser. No. 63/635,101, filed Apr. 17, 2024, the entire contents of each of which are incorporated herein by reference.

### FIELD

The disclosure relates generally to medical device systems and, more particularly, medical device systems configured to detect falls and cardiac activity.

### BACKGROUND

Medical devices may be used to monitor physiological signals of a patient. For example, some medical devices are configured to sense cardiac electrocardiogram (ECG) signals indicative of the electrical activity of the heart via electrodes. Some medical devices are additionally or alternatively configured to sense other signals, such as heart sound signals indicative of the mechanical activity of the heart via a motion or vibration sensor, such as an accelerometer or microphone. Some medical devices may be configured to deliver a therapy in conjunction with or separate from the monitoring of physiological signals.

### SUMMARY

In general, this disclosure is directed to techniques for identifying a fall event of a patient as a cardiac fall event, generating a personalized ECG signal parameter fall event threshold, outputting an indication the patient is at risk of a fall event within a period of time and/or outputting a degree of risk of a fall event within a period of time. Processing circuitry may implement the techniques to determine a patient suffered a fall event. Processing circuitry may determine the patient suffered the fall event based on a sensed accelerometer signal. In response to the determination that the patient suffered the fall event, processing circuitry may determine whether one or more parameters of the ECG signal during a first period of time indicate a presence of a cardiac event, the first period of time being before a time the fall event occurs. In response to a determination that the one or more parameters of the ECG signal during the first period of time indicate the presence of a cardiac event, processing circuitry may identify the fall event as a cardiac fall event. In some examples, in response to a determination that the one or more parameters of the ECG signal during the first period of time indicate the presence of a cardiac event, processing circuitry may generate an ECG signal parameter fall event threshold based on the one or more parameters of the ECG signal during the first period of time.

In some examples, processing circuitry may determine whether one or more parameters of an ECG signal during a second period of time satisfies the ECG parameter fall event threshold, the second period of time being after the time the fall event occurs. In response to a determination that the one or more parameters of the ECG signal during the second period of time satisfies the ECG parameter fall event threshold, processing circuitry may output an indication the patient is at risk of another fall event within a third period of time and/or output an indication of a degree of risk of another fall event by the patient within a third period of time, the third period of time being after the output of the indication. In some examples, the third period of time may end within one hour after the output of the indication. In some examples, the cardiac event is an arrhythmia.

The combination of an implantable medical device sensing the ECG signal with the processing circuitry identifying the fall event as a cardiac fall event based on the ECG signal may help a medical system identify fall events as cardiac fall events with greater speed and specificity and sensitivity. In some examples, by generating an ECG signal parameter fall event threshold based on the one or more parameters of the ECG signal during the first period of time the system, insertable cardiac monitor (ICM), and method techniques described herein may provide a personalized ECG signal parameter fall event threshold that may be used to determine and/or warn when a patient is at risk of future cardiac fall event with greater specificity and sensitivity and with greater speed. A system or device determining and/or warning when a patient is at risk of future cardiac fall event more quickly and with greater specificity and sensitivity, a patient may be more likely to take proper precautions based upon receiving such indications or warnings of risk of future cardiac fall event from the respective system or device.

In some examples, the system, device, and/or method techniques described herein outputting an indication that the patient is at risk of a fall event and/or output an indication of a degree the patient is at suffering a fall event within a period of time, such as an hour, may inform a patient of a pending potential catastrophic fall event so the patient may act to reduce the chances of the fall event from occurring and/or reduce the potential harm of the fall event. For example, a patient may sit down, seek medical help, contact an emergency responder, contact a friend/family member, etc. in response to receiving an indication that the patient is at risk of a fall event and/or output an indication of a degree the patient is at suffering a fall event within a period of time.

In one example, this disclosure describes a system comprising an implantable medical device comprising a plurality of electrodes configured to sense an electrocardiogram (ECG) signal of a patient; and processing circuitry configured to: determine the patient suffered a fall event; in response to the determination that the patient suffered the fall event, determine whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identify the fall event as a cardiac fall event.

In another example, this disclosure describes an ICM comprising: a housing configured for subcutaneous implantation within a patient; a plurality of electrodes on the housing, wherein the ICM is configured to sense an electrocardiogram (ECG) signal of the patient via the plurality of electrodes; and processing circuitry configured to: determine the patient suffered a fall event; in response to the determination that the patient suffered the fall event, determine whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identify the fall event as a cardiac fall event.

In another example, this disclosure describes a method comprising: receiving an electrocardiogram (ECG) signal of a patient; receiving a sensed accelerometer signal; determining the patient suffered a fall event based on the sensed accelerometer signal; in response to the determination that the patient suffered the fall event, determining whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identifying the fall event as a cardiac fall event.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient.

FIG. 2 is a functional block diagram illustrating an example configuration of the implantable medical device (IMD) of the medical system of FIG. 1.

FIG. 3 is a conceptual side-view diagram illustrating an example configuration of the IMD of FIGS. 1 and 2.

FIG. 4A is a conceptual side-view diagram illustrating an example configuration of the IMD of FIGS. 1-2.

FIG. 4B is a conceptual side-view diagram illustrating an example configuration of the IMD of FIGS. 1-3.

FIG. 5 is a functional block diagram illustrating an example configuration of the external device of FIG. 1.

FIG. 6 is a block diagram illustrating an example system that includes an access point, a network, external computing devices, such as a server, and one or more other computing devices, which may be coupled to the IMD and external device of FIGS. 1-5.

FIG. 7 is a flow diagram illustrating an example technique for operating a system to output an indication a patient is at risk of a fall event within a period of time.

FIG. 8 is a flow diagram illustrating an example technique for operating a system to output an indication of a degree of risk of a fall event within a period of time.

FIG. 9 is a conceptual diagram illustrating an example training process for an artificial intelligence model, in accordance with examples of the current disclosure.

FIG. 10 is a conceptual diagram illustrating an example ML model configured to determine whether one or more parameters of an ECG signal indicate a presence of a cardiac event and/or determine a degree of risk fall event within a period of time based on one or more parameter values of an ECG signal.

Like reference characters denote like elements throughout the description and figures.

### DETAILED DESCRIPTION

A variety of types of medical devices sense cardiac data. In some examples, cardiac data may include one or more of electrocardiogram (ECG or EKG) signals, electrogram signals, and/or heart sound signals. In some examples, a variety of types of medical device may sense patient activity data, such as via an accelerometer.

The electrodes used by IMDs to sense cardiac data are typically integrated with a housing of the IMD and/or coupled to the IMD via one or more elongated leads. Example IMDs that monitor cardiac data include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic, Inc. Some IMDs that do not provide therapy, e.g., implantable patient monitors, sense cardiac data. One example of such an IMD is the Reveal LINQ™ and LINQ II™ Insertable Cardiac Monitors (ICMs), available from Medtronic, Inc, which may be inserted subcutaneously. Such IMDs may facilitate relatively longer-term monitoring of patients during normal daily activities, and may periodically transmit collected data to a network service, such as the Medtronic Carelink™ Network.

Any medical device configured to sense a cardiac data via implanted or external electrodes, including the examples identified herein, may implement the techniques of this disclosure for identifying a fall event of a patient as a cardiac fall event. For example, circuitry of at least one of an IMD or a remote computing device may determine a patient suffered a fall event. In some examples, a fall event may include when a patient moves from an upright or vertical position to a non-upright posture or horizontal posture. In some examples, a fall event may include when a patient moves from an upright or vertical position to a non-upright posture or horizontal posture because, at least in part, of gravitational forces. In some examples, a fall event may include moving from a non-upright or horizontal posture to another non-upright or horizontal posture because, at least in part, of gravitational forces, e.g. when a patient rolls or of bed from a lying posture and lands on the floor in a non-upright posture. In some examples, a fall event may include a patient making impact with a surface, such as the ground or floor, because, at least in part, of gravitational forces. In some examples, circuitry may determine the patient suffered the fall event based on a sensed accelerometer signal. In response to the determination that the patient suffered the fall event, circuitry may determine whether one or more parameters of the ECG signal during a first period of time indicate a presence of a cardiac event, the first period of time being before a time the fall event occurs. In response to a determination that the one or more parameters of the ECG signal during the first period of time indicate the presence of a cardiac event, circuitry may identify the fall event as a cardiac fall event. In some examples, circuitry may be able to provide a personalized identification of whether a fall event is a cardiac event that may be provided automatically and without having to see a clinician. The combination of the implantable medical device sensing the ECG signal with the circuitry identifying the fall event as a cardiac fall event based on the ECG signal may help a medical system 2 to identify fall events as cardiac fall events with greater speed and specificity and sensitivity.

In some examples, in response to a determination that the one or more parameters of the ECG signal during the first period of time indicate the presence of a cardiac event, circuitry may generate an ECG signal parameter fall event threshold based on the parameters of the ECG signal during the first period of time. In some examples, a duration of the first period of time may be up to 15 minutes. In some examples, by generating an ECG signal parameter fall event threshold based on the one or more parameters of the ECG signal during the first period of time the system, ICM, and method techniques described herein may improve the system, ICM, and/or method by helping provide a personalized ECG signal parameter fall event threshold that may be used to determine and/or warn when a patient is at risk of future cardiac fall event with greater specificity and sensitivity and with greater speed. The system, ICM, and method techniques described herein determining and/or warning when a patient is at risk of future cardiac fall event more quickly and with greater specificity and sensitivity, a patient may be more likely to take proper precautions based upon receiving such indications or warnings of risk of future cardiac fall event.

In some examples, circuitry may determine whether one or more parameters of an ECG signal during a second period of time satisfies the ECG parameter fall event threshold, the second period of time being after the time the fall event occurs. In some examples, in response to a determination that the one or more parameters of the ECG signal during the second period of time satisfies the ECG parameter fall event threshold, circuitry may output an indication the patient is at risk of a fall event within a third period of time, the third period of time being after the output of the indication. In some examples, circuitry may determine whether one or more parameters of an ECG signal during a second period of time satisfies the ECG parameter fall event threshold, the second period of time being after the time the fall event occurs. In some examples, in response to a determination that the one or more parameters of the ECG signal during the second period of time satisfies the ECG parameter fall event threshold, circuitry may output an indication of a degree of risk of a fall event by the patient within a third period of time, the third period of time being after the output of the indication. In some examples, the third period of time may end within one hour after the output of the indication. In some examples, the cardiac event is an arrhythmia.

In some examples, the system, ICM, and method techniques described herein outputting an indication that the patient is at risk of a fall event and/or output an indication of a degree the patient is at suffering a fall event within a period of time, such as an hour, may inform a patient of a pending potential catastrophic fall event so the patient may act to reduce the chances of the fall event from occurring and/or reduce the potential harm of the fall event. For example, a patient may sit down, seek medical help, contact an emergency responder, contact a friend/family member, etc. in response to receiving an indication that the patient is at risk of a fall event and/or receiving an indication of a degree the patient is at suffering a fall event within a period of time.

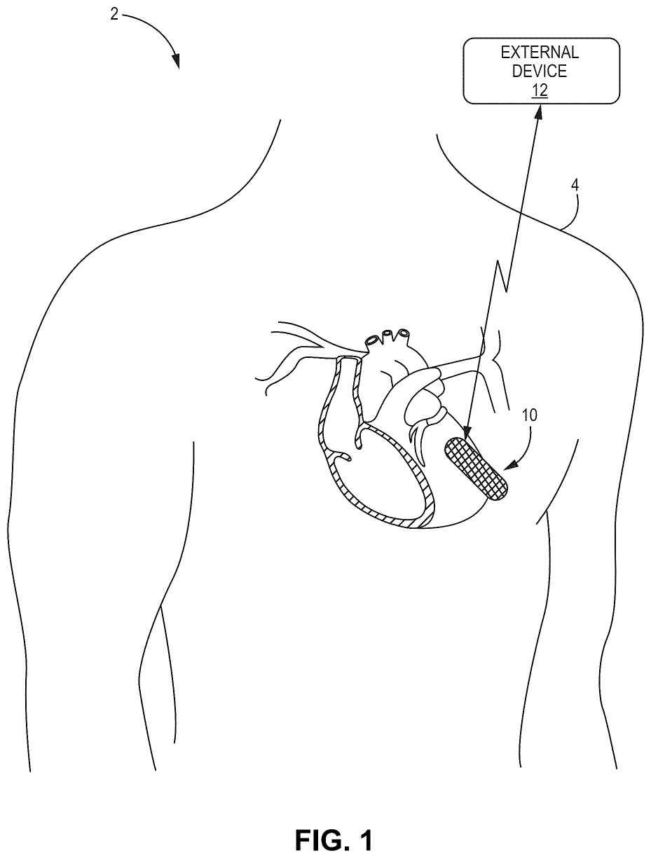

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac data via the plurality of electrodes. In some examples, IMD 10 takes the form of the Reveal LINQ™ or LINQ II ICM™, or another ICM similar to, e.g., a version or modification of, the LINQ™ ICMS.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, smartphone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

In some examples, external device 12 may be or additionally include wearable computing device. A wearable computing device may include electrodes and other sensors to sense physiological signals of patient 4, and may collect and store physiological data and detect episodes based on such signals. Wearable computing device may be incorporated into the apparel of patient 4, such as within clothing, shoes, eyeglasses, a watch or wristband, a hat, etc. In some examples, a wearable device may be a smartwatch or other accessory or peripheral for external device 12, for example when external device 12 is a smartphone or tablet.

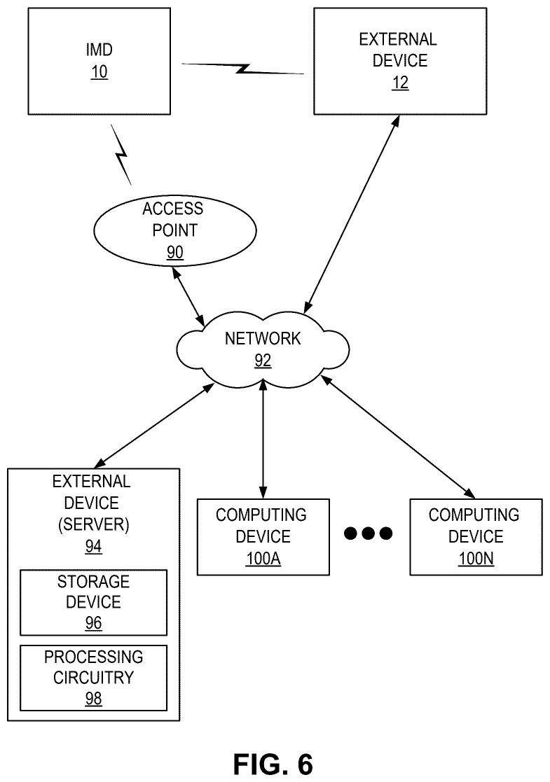

External device 12 may be used to configure operational parameters for IMD 10. External device 12 may be used to retrieve data from IMD 10. The retrieved data may include values of physiological parameters measured by IMD 10, indications of fall events detected by IMD 10, and/or other physiological signals recorded by IMD 10. For example, external device 12 may retrieve an ECG signal from IMD 10 and/or an accelerometer signal from IMD 10. In some examples, external device 12 may determine a patient suffered a fall event. In some examples, external device 12 may determine the patient suffered the fall event based on an accelerometer signal received from the IMD 10. In some examples, in response to the determination that the patient suffered the fall event, external device 12 may determine whether one or more parameters of the ECG signal during a first period of time indicate a presence of a cardiac event based on an ECG signal received from the IMD 10, the first period of time being before a time the fall event occurs. In some examples, external device 12 may also retrieve an ECG signal and/or an accelerometer signal recorded by IMD 10 due to IMD 10 determining that patient 4 suffered a fall event or in response to a request to retrieve the ECG signal and/or the accelerometer signal from patient 4 or another user. As discussed in greater detail below with respect to FIG. 6, one or more remote computing devices may interact with IMD 10 in a manner similar to external device 12, e.g., to program IMD 10 and/or retrieve data from IMID 10, via a network.

Processing circuitry of medical system 2, e.g., of IMD 10, external device 12, and/or of one or more other computing devices, may be configured to perform the example techniques of this disclosure for identifying a fall event of a patient as a cardiac fall event. For example, processing circuitry of medical system 2 may determine a patient suffered a fall event. In some examples, IMD 10 may include an accelerometer within the housing configured to sense an accelerometer signal. In some examples, processing circuitry of medical system 2 may determine the patient suffered the fall event based on a sensed accelerometer signal. In response to the determination that the patient suffered the fall event, processing circuitry of medical system 2 may determine whether one or more parameters of the ECG signal during a first period of time indicate a presence of a cardiac event, the first period of time being before a time the fall event occurs. In some examples, parameters of the ECG signal that processing circuitry of medical system 2 may use to determine an indication of a cardiac event may include one or more a cardiac pause duration, a duration of arrhythmia, a duration of ventricular tachycardia (VT), a duration of ventricular fibrillation (VF), QRS morphology, T-wave morphology, changes in QRS morphology, QRS width, changes in T-wave morphology, QT interval, changes in QT interval, ST elevation, changes in ST elevation, indication of heart rate, changes in heart rate, variability of heart rate, premature ventricular contraction (PVC) burden, PVC morphology, occurrence of couplets, occurrence of triplets, type of PVC patterns, such as bigeminy or trigeminy, and/or indication of blood pressure.

For example, processing circuitry of medical system 2 may determine that the ECG signal during a first period of time includes a cardiac pause duration greater than a pause duration threshold, such as between 8-10 seconds, 6-10 seconds, or 4-12 seconds, and determine the one or more parameters of the ECG signal during a first period of time indicate a presence of a cardiac event based on the ECG signal during a first period of time includes a cardiac pause duration greater than a pause duration threshold.

In response to a determination that the one or more parameters of the ECG signal during the first period of time indicate the presence of a cardiac event, processing circuitry of medical system 2 may identify the fall event as a cardiac fall event. In some examples, in response to a determination that the one or more parameters of the ECG signal during the first period of time indicate the presence of a cardiac event, processing circuitry of medical system 2 may generate an ECG signal parameter fall event threshold based on the one or more parameters of the ECG signal during the first period of time. In some examples, a duration of the first period of time may be up to 20 minutes. In some examples, a duration of the first period of time may be up to 15 minutes.

For example, medical system 2 may determine that the ECG signal during a first period of time includes a cardiac pause duration, such as 9 seconds, which is greater than an initial pause duration threshold, such as 4 seconds, which may be pre-determined. Processing circuitry of medical system 2 may then generate a personalized pause duration threshold (e.g., an ECG signal parameter fall event threshold) to be between 8-9 seconds based on the cardiac pause duration during first period of time before the fall event having a duration of 9 seconds.

In some examples, processing circuitry of medical system 2 may determine whether one or more parameters of an ECG signal during a second period of time satisfies the ECG parameter fall event threshold, the second period of time being after the time the fall event occurs. In some examples, in response to a determination that the one or more parameters of the ECG signal during the second period of time satisfies the ECG parameter fall event threshold, processing circuitry of medical system 2 may output an indication the patient is at risk of a fall event within a third period of time, the third period of time being after the output of the indication. In some examples, processing circuitry of medical system 2 may determine whether one or more parameters of an ECG signal during a second period of time satisfies the ECG parameter fall event threshold, the second period of time being after the time the fall event occurs. In some examples, in response to a determination that the parameters of the ECG signal during the second period of time satisfies the ECG parameter fall event threshold, processing circuitry of medical system 2 may output an indication of a degree of risk of a fall event by the patient within a third period of time, the third period of time being after the output of the indication. In some examples, the third period of time may end within one hour after the output of the indication. In some examples, the cardiac event is an arrhythmia.

Although described in the context of examples in which IMD 10 that senses the cardiac data comprises an insertable cardiac monitor, example systems including one or more implantable or external devices of any type configured to sense cardiac data may be configured to implement the techniques of this disclosure.

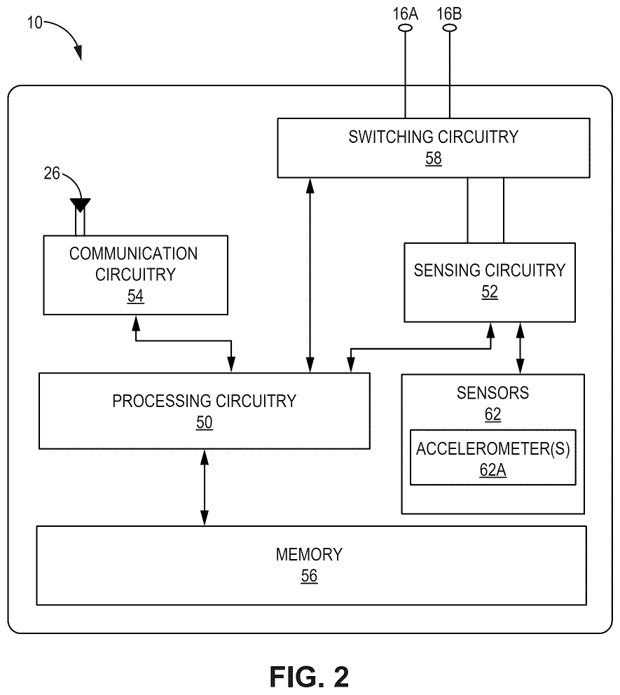

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, sensors 62, and accelerometer(s) 62A. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense an ECG signal, as controlled by processing circuitry 50. Sensing circuitry 52 may sense signals from electrodes 16, e.g., to produce an ECG signal, in order to facilitate monitoring the electrical activity of the heart. Sensing circuitry 52 may include components/modules for converting the raw ECG signal to a processed ECG signal that can be analyzed to detect sense events. Sensing circuitry 52 also may monitor signals from sensors 62, such as one or more accelerometers 62A. As described in commonly-assigned U.S. patent application Ser. No. 16/879,499, entitled, “MEDICAL DEVICE FOR FALL DETECTION” to Michelle M. Galarneau, incorporated herein by reference in its entirety, processing circuitry 50 may determine, based at least in part on sensor data received from one or more accelerometers 62A, an indication of one or more fall events by patient 4. For example, one or more accelerometers 62A and/or other body-position sensors may indicate a sudden deviation (an above-threshold acceleration away) from an “upright” body posture, indicative of a fall event. Additionally or alternatively, since loss of consciousness and/or falls may be caused by a sudden drop in blood pressure, processing circuitry 50 may determine an indication of a fall based on sensor data received from IMD 10 indicating such a sudden drop. In some examples, sensors 62 may additionally or alternatively include one or more microphones and/or other vibration/motion sensors. In some examples, sensing circuitry 52 may include one or more filters and amplifiers for filtering and amplifying signals received from electrodes 16 and/or sensors 62.

Sensing circuitry 52 and/or processing circuitry 50 may be configured to detect cardiac depolarizations (e.g., P-waves of atrial depolarizations or R-waves of ventricular depolarizations) when the ECG signal amplitude crosses a sensing threshold. An ECG signal may include P-waves (depolarization of the atria), R-waves (depolarization of the ventricles), and T-waves (repolarization of the ventricles), among other events. Sensing circuitry 52 and/or processing circuitry 50 may be configured to detect one or more features of the P-waves, R-waves, and/or T-waves in an ECG signal. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining inter-depolarization intervals, heart rate, and detecting arrhythmias, such as tachyarrhythmias and asystole.

Sensing circuitry 52 may also provide one or more digitized ECG signals and/or heart sound beat signals to processing circuitry 50 for analysis, e.g., for use in cardiac rhythm discrimination. Sensing circuitry 52 may include one or more detection channels, each of which may include an amplifier. The detection channels may be used to sense cardiac data, such as an ECG signal. Some detection channels may detect events, such as R-waves, P-waves, and T-waves and provide indications of the occurrences of such events to processing circuitry 50. One or more other detection channels may provide the signals to an analog-to-digital converter, for conversion into a digital signal for processing or analysis by processing circuitry 50.

In some examples, processing circuitry 50 may store the digitized cardiac ECG signal and/or accelerometer signal in storage device 56. Processing circuitry 50 of IMD 10, and/or processing circuitry of another device that retrieves data from IMD 10, may analyze the cardiac ECG signal to determine whether one or more parameters of the ECG signal during a first period of time indicate a presence of a cardiac event, the first period of time being before a time the fall event occurs, as describe herein. In some examples, processing circuitry 50 of IMD 10, and/or processing circuitry of another device that retrieves data from IMD 10, may analyze the cardiac ECG signal to generate an ECG signal parameter fall event threshold based on the one or more parameters of the ECG signal during the first period of time. In some examples, processing circuitry 50 of IMD 10, and/or processing circuitry of another device that retrieves data from IMD 10, may analyze a cardiac ECG signal during a second period of time to determine whether one or more parameters of an ECG signal during a second period of time satisfy the ECG parameter fall event threshold.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink™ Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include fall event detection indications, digitized ECG signals, accelerometer signals, indications of ECG signal parameter fall event threshold, indications patient is at risk of a fall event within a period of time, and/or indications of a degree of risk of a fall event by the patient is at risk within a period of time, as examples.

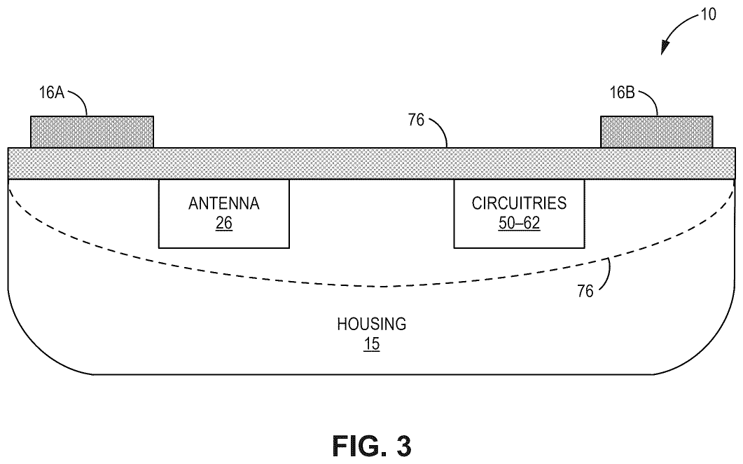

FIG. 3 is a conceptual side-view diagram illustrating an example configuration of IMD 10 of FIGS. 1 and 2. In the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

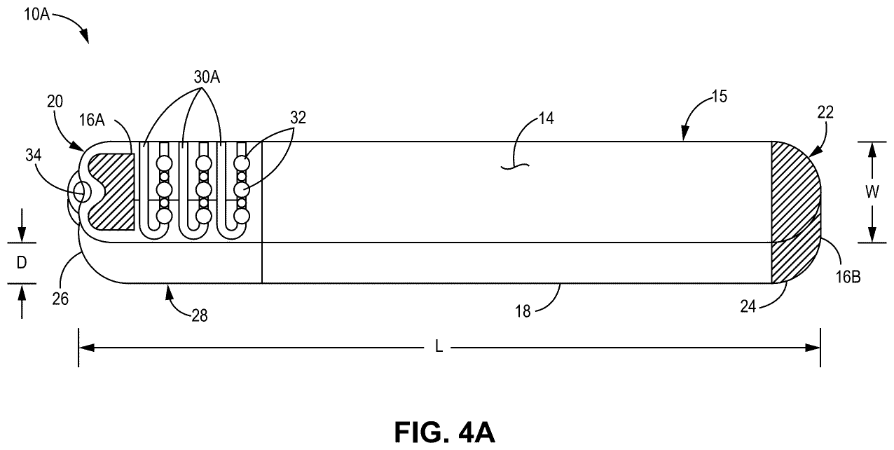

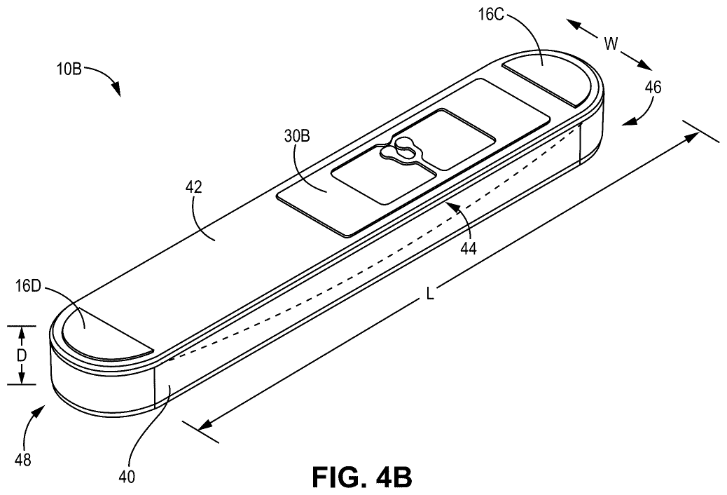

FIG. 4A is a conceptual drawing illustrating an IMD 10A, which may be an example configuration of IMD 10 of FIGS. 1-3 as an ICM. In the example shown in FIG. 4A, IMD 10A may be embodied as a monitoring device having housing 15, proximal electrode 16A and distal electrode 16B. Housing 15 may further comprise first major surface 14, second major surface 18, proximal end 20, and distal end 22. Housing 15 encloses electronic circuitry located inside the IMD 10A and protects the circuitry contained therein from body fluids. Electrical feedthroughs provide electrical connection of electrodes 16A and 16B.

In the example shown in FIG. 4A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during and after insertion. For example, the device shown in FIG. 4A includes radial asymmetries (notably, the rectangular shape) along the longitudinal axis that maintains the device in the proper orientation following insertion. For example, the spacing between proximal electrode 16A and distal electrode 16B may range from 30 millimeters (mm) to 55 mm, 35 mm to 55 mm, and from 40 mm to 55 mm and may be any range or individual spacing from 25 mm to 60 mm. In addition, IMD 10A may have a length L that ranges from 30 mm to about 70 mm. In other examples, the length L may range from 5 mm to 60 mm, 15 mm to 50 mm, 40 mm to 60 mm, 45 mm to 60 mm and may be any length or range of lengths between about 5 mm and about 80 mm. In addition, the width W of major surface 14 may range from 5 mm to 15 mm, 3 mm to 10 mm, and may be any single or range of widths between 3 mm and 15 mm. The thickness of depth D of IMD 10A may range from 2 mm to 9 mm. In other examples, the depth D of IMD 10A may range from 2 mm to 5 mm, may range from 5 mm to 15 mm, and may be any single or range of depths from 2 mm to 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic cm.

In the example shown in FIG. 4A, once inserted within the patient, the first major surface 14 faces outward, toward the skin of the patient while the second major surface 18 is located opposite the first major surface 14. In addition, in the example shown in FIG. 4A, proximal end 20 and distal end 22 are rounded to reduce discomfort and irritation to surrounding tissue once inserted under the skin of the patient. IMD 10A, including instrument and method for inserting IMD 10 is described, for example, in U.S. Patent Publication No. 2014/0276928, incorporated herein by reference in its entirety.

Proximal electrode 16A and distal electrode 16B are used to sense cardiac data, e.g. ECG signals, intra-thoracically or extra-thoracically, which may be sub-muscularly or subcutaneously. Cardiac data, such as ECG signals, may be stored in a memory of IMD 10A, and data may be transmitted via integrated antenna 30A to another medical device, which may be another implantable device or an external device, such as external device 12. In some example, electrodes 16A and 16B may additionally or alternatively be used for sensing any bio-potential signal of interest, which may be, for example, an ECG, EGM, EEG, EMG, or a nerve signal, from any implanted location.

In the example shown in FIG. 4A, proximal electrode 16A is in close proximity to the proximal end 20 and distal electrode 16B is in close proximity to distal end 22. In this example, distal electrode 16B is not limited to a flattened, outward facing surface, but may extend from first major surface 14 around rounded edges 24 and/or end surface 25 and onto the second major surface 18 so that the electrode 16B has a three-dimensional curved configuration. In some examples, electrode 16B is an uninsulated portion of a metallic, e.g., titanium, part of housing 15.

In the example shown in FIG. 4A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 4A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 4A, proximal end 20 includes a header assembly 28 that includes one or more of proximal electrode 16A, integrated antenna 30A, anti-migration projections 32, and/or suture hole 34. Integrated antenna 30A is located on the same major surface (i.e., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 15 of IMD 10A. In the example shown in FIG. 4A, anti-migration projections 32 are located adjacent to integrated antenna 30A and protrude away from first major surface 14 to prevent longitudinal movement of the device. In the example shown in FIG. 4A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 4A, header assembly 28 includes suture hole 34, which provides another means of securing IMD 10A to the patient to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 4B is a perspective drawing illustrating another IMD 10B, which may be another example configuration of IMD 10 from FIGS. 1-3. IMD 10B of FIG. 4B may be configured substantially similarly to IMD 10A of FIG. 4A, with differences between them discussed herein.

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 4B, the housing of IMD 10B defines a length L, a width W and thickness or depth D and is in the form of an elongated rectangular prism wherein the length L is much larger than the width W, which in turn is larger than the depth D, similar to IMD 10A of FIG. 4B. For example, the spacing between proximal electrode 16C and distal electrode 16D may range from 30 mm to 50 mm, from 35 mm to 45 mm, or be approximately 40 mm. In addition, IMD 10B may have a length L that ranges from 30 mm to about 70 mm. In other examples, the length L may range from 5 mm to 60 mm, 40 mm to 60 mm, 45 mm to 55 mm, or be approximately 45 mm. In addition, the width W may range from 3 mm to 15 mm, such as approximately 8 mm.

The thickness of depth D of IMD 10B may range from 2 mm to 15 mm, from 3 to 5 mm, or be approximately 4 mm. IMD 10B may have a volume of three cubic centimeters (cm) or less, or 1.5 cubic cm or less, such as approximately 1.4 cubic cm.

In the example shown in FIG. 4B, once inserted subcutaneously within the patient, outer surface of cover 42 faces outward, toward the skin of the patient. In addition, as shown in FIG. 4B, proximal end 46 and distal end 48 are rounded to reduce discomfort and irritation to surrounding tissue once inserted

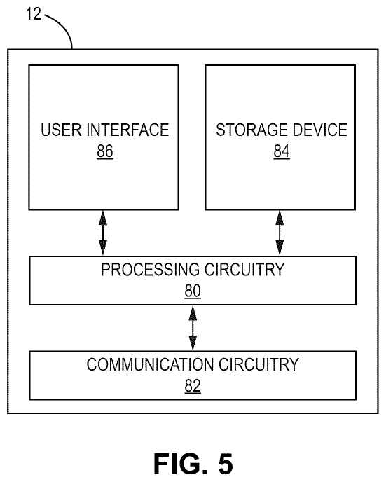

FIG. 5 is a block diagram illustrating an example configuration of components of external device 12. In the example of FIG. 5, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected cardiac data (e.g., digitized ECG signals, and/or digitized heart sound beat signals) to external device 12. In turn, external device 12 may receive the collected cardiac data from IMD 10 and store the collected cardiac data in storage device 84. Processing circuitry 80 may implement any of the techniques described herein to analyze ECG signal(s), and/or accelerometer signals received from IMD 10, e.g., to identify a fall event of patient 4 as a cardiac fall event, generate an ECG signal parameter fall event threshold based on one or more parameters of the ECG signal during a period of time before the fall event, output an indication the patient is at risk of a fall event within a period of time, and/or output an indication of a degree of risk of a fall event by the patient a third period of time.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac data, ECG signals, ECG signal features, accelerometer signals, indications of ECG signal parameter fall event threshold, indications patient is at risk of a fall event within a period of time, and/or indications of a degree of risk of a fall event by the patient is at risk within a period of time. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 6 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 6, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as digitized ECG signals, digitized accelerometer signals, indications of ECG signal parameter fall event threshold, indications patient is at risk of a fall event within a period of time, and/or indications of a degree of risk of a fall event by the patient is at risk within a period of time, to access point 90. Access point 90 may then communicate the retrieved cardiac data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 6 may be implemented with general network technology and functionality, which may be similar to that provided by the Medtronic CareLink™ Network. In some examples, server 94 may communicate with computing device 100 via network 92. For example, server 94 may communicate an analysis of data, such as determination of a position of an S2, S3, or S4 window or HF information determined based on heart sound features, to computing device 100, external device 12, or any other computing device via network 92. For example, server 94 may communicate that a patient has suffered fall event or is at risk of suffering a fall event within a period of time or communicate a quantification of a risk the patient has of suffering a fall event within a period of time of the patient to computing device 100, external device 12, or any other computing device via network 92.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition, such as when patient 4 is at a high risk of suffering a cardiac fall event within an upcoming period of time. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a risk of patient suffering a fall event within an upcoming period of time, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 6, server 94 includes a storage device 96, e.g., to store data retrieved from IMD 10, and processing circuitry 98. Although not illustrated in FIG. 6 computing devices 100 may similarly include a storage device and processing circuitry.

Processing circuitry 98 may include one or more processors that are configured to implement functionality and/or process instructions for execution within server 94. For example, processing circuitry 98 may be capable of processing instructions stored in storage device 96. Processing circuitry 98 may include or be coupled to communication circuitry that may include any suitable hardware, firmware, software or any combination thereof for communicating with another device. In some examples, a description of processing circuitry 98 outputting a signal, such as a classification, may include processing circuitry 98 causing communication circuitry of server 94 to output the signal. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 100 may implement any of the techniques described herein to analyze ECG signals and/or accelerometer signals received from IMD 10, e.g., to identify a fall event of patient 4 as a cardiac fall event, generate an ECG signal parameter fall event threshold based on one or more parameters of the ECG signal during a period of time before the fall event, output an indication the patient is at risk of a fall event within a period of time, and/or output an indication of a degree of risk of a fall event by the patient a third period of time.

Storage device 96 may include a computer-readable storage medium or computer-readable storage device. In some examples, storage device 96 may be referred to as a memory. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

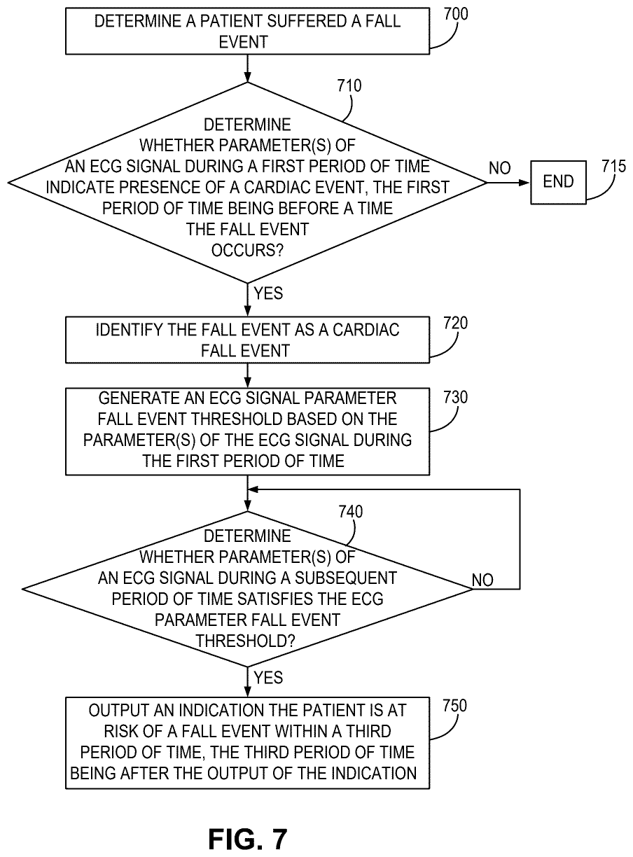

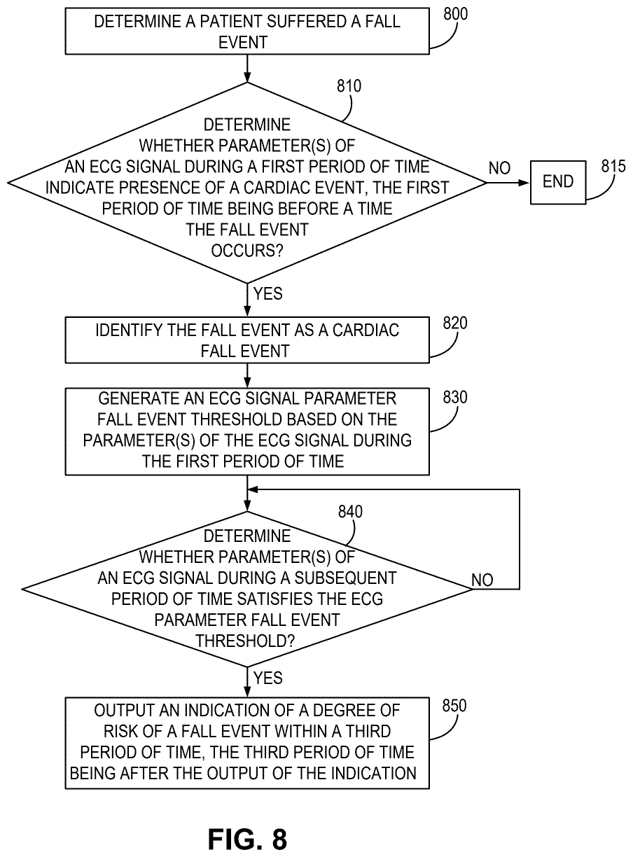

Although the techniques for to identify a fall event of patient 4 as a cardiac fall event, generate an ECG signal parameter fall event threshold based on one or more parameters of the ECG signal during a period of time before the fall event, output an indication the patient is at risk of a fall event within a period of time, and/or output an indication of a degree of risk of a fall event by the patient a third period of time, are described herein primarily (e.g., with respect to FIGS. 7-8) as being performed by processing circuitry 50 of IMD 10, such techniques may be performed, in whole or part, by processing circuitry of any one or more devices of system 2, processing circuitry 98 of server 94, processing circuitry of one or more computing devices 100, or processing circuitry 80 of external device 12.

FIG. 7 is a flow diagram illustrating an example technique for operation of a medical system 2 or IMD 10 to identify a fall event of a patient as a cardiac fall event, generate a personalized ECG signal parameter fall event threshold, and output an indication the patient is at risk of a fall event within a third period of time, the third period of time being after the output of the indication. As indicated by FIG. 7, processing circuitry 50 may determine a patient suffered a fall event (700). In some examples, processing circuitry 50 may determine a patient suffered a fall event based, at least in part, on an accelerometer signal sensed by an accelerometer 62A positioned in IMD 10. Processing circuitry 50 may determine whether one or more parameters of an ECG signal during a first period of time indicate a presence of a cardiac event, the first period of time being before a time the fall event occurs (710). In some examples, the cardiac event is an arrhythmia. In some examples, the ECG signal may be sensed via a plurality of electrodes 16 of IMD 10. For example, processing circuitry 50 may determine whether one or more parameters of an ECG signal during a first period of time indicate a presence of a cardiac event in response to the determination that the patient suffered the fall event. In some examples, parameters of the ECG signal may include one or more of a cardiac pause duration, a duration of arrhythmia, a duration of ventricular tachycardia (VT), a duration of ventricular fibrillation (VF), QRS morphology, T-wave morphology, changes in QRS morphology, changes in T-wave morphology, QT interval, changes in QT interval, ST elevation, changes in ST elevation, indication of heart rate, changes in heart rate, heart rate variability, and/or indication of blood pressure.

In response to a determination that the one or more parameters of the ECG signal during the first period of time does not indicate the presence of a cardiac event (e.g. “NO”), processing circuitry 50 may end the operation (715). In response to a determination that the one or more parameters of the ECG signal during the first period of time indicate the presence of a cardiac event (e.g. “YES”), processing circuitry 50 may identify the fall event as a cardiac fall event (720). For example, processing circuitry 50 may determine that the ECG signal during a first period of time includes a cardiac pause duration greater than a pre-determined pause duration threshold, such as between 8-10 seconds, 6-10 seconds, or 4-12 seconds, and determine the one or more parameters of the ECG signal during a first period of time indicate a presence of a cardiac event based on the ECG signal during a first period of time includes a cardiac pause duration greater than a pause duration threshold. In some examples, processing circuitry 50 may be able to provide a personalized identification of whether a fall event is a cardiac event that may be provided automatically and without having to see a clinician. The combination of IMD 10 sensing the ECG signal with the processing circuitry 50 identifying the fall event as a cardiac fall event based on the ECG signal may help processing circuitry 50 identify fall events as cardiac fall events with greater speed and specificity and sensitivity.

Processing circuitry 50 may generate an ECG signal parameter fall event threshold based on the one or more parameters of the ECG signal during the first period of time (730). In some examples, a duration of the first period of time may be up to 15 minutes. In some examples, a duration of the first period of time may be up to 20 minutes. In some examples, processing circuitry 50 may generate an ECG signal parameter fall event threshold based on the one or more parameters of the ECG signal during the first period of time in response to a determination that the one or more parameters of the ECG signal during the first period of time indicate the presence of a cardiac event.

For example, processing circuitry 50 may determine that the ECG signal during a first period of time includes a cardiac pause duration, such as 9 seconds, which is greater than an initial pause duration threshold, such as 4 seconds, which may be pre-determined (e.g., the one or more parameters of the ECG signal indicate the presence of a cardiac event). Processing circuitry 50 may then generate a personalized pause duration threshold (e.g., an ECG signal parameter fall event threshold), such as being between 8-9 seconds, based on the cardiac pause duration during first period of time before the fall event having a duration of 9 seconds. Some other examples of ECG signal parameter fall event thresholds may be based on VT duration, VF duration, QT interval, ST elevation, QRS morphology, presence of PVCs, morphology of PVC, PVC burden, occurrence of couplet, occurrence of triplet, and/or T-wave morphology.

In some examples, processing circuitry 50 generating an ECG signal parameter fall event threshold based on the one or more parameters of the ECG signal during the first period of time may improve the system 2 and/or IMD 10 by helping provide a personalized ECG signal parameter fall event threshold that may be used to determine and/or warn when a patient is at risk of future cardiac fall event with greater specificity and sensitivity and with greater speed. Since the system 2 or IMD 10 may determine and/or warn when a patient is at risk of future cardiac fall event more quickly and with greater specificity and sensitivity, a patient may be more likely to take proper precautions based upon receiving such indications or warnings of risk of future cardiac fall event.

Processing circuitry 50 may determine whether one or more parameters of an ECG signal during a subsequent period of time (e.g., second period of time) satisfies the ECG parameter fall event threshold, the second period of time being after the time the fall event occurs (740). In response to a determination that the one or more parameters of the ECG signal during a subsequent period of time satisfies the ECG parameter fall event threshold (e.g., “NO”), processing circuitry 50 may return to operation 740 to determine whether one or more parameters of an ECG signal during an additional subsequent period of time satisfies the ECG parameter fall event threshold. In response to a determination that the one or more parameters of the ECG signal during the subsequent period of time (e.g., second period of time) satisfies the ECG parameter fall event threshold (e.g., “YES”), processing circuitry 50 may output an indication the patient is at risk of a fall event within a third period of time, the third period of time being after the output of the indication (750). In some examples, the third period of time may be after the output of the indication. In some examples, the third period of time may end within one hour after the output of the indication. In some examples, the third period of time may end within 30 minutes after the output of the indication. In some examples, the third period of time may end within 6 hours after the output of the indication. For example, if processing circuitry 50 determines an ECG signal during the second period of time includes a pause duration that satisfies the generated personalized pause duration threshold (e.g., an ECG signal parameter fall event threshold), processing circuitry 50 may output an indication the patient is at risk of a fall event within a third period of time.

In some examples, the system 2 or IMD 10 outputting an indication that the patient is at risk of a fall event a period of time, such as an hour, may inform a patient of a pending potential catastrophic fall event so the patient may act to reduce the chances of the fall event from occurring and/or reduce the potential harm of the fall event. For example, a patient may sit down, seek medical help, contact an emergency responder, contact a friend/family member, etc. in response to receiving an indication that the patient is at risk of a fall event within a period of time.

FIG. 8 is a flow diagram illustrating an example technique for operation of a medical system 2 or IMD 10 to identify a fall event of a patient as a cardiac fall event, generate a personalized ECG signal parameter fall event threshold, and output an indication of a degree of risk of a fall event within a third period of time, the third period of time being after the output of the indication. As indicated by FIG. 8, items 800-840 are similar to items 700-740 shown in FIG. 7 and discussed above. In response to a determination that the one or more parameters of the ECG signal during the subsequent period of time (e.g., second period of time) satisfies the ECG parameter fall event threshold (e.g., “YES”), processing circuitry 50 may output an indication of a degree of risk of a fall event within a third period of time, the third period of time being after the output of the indication (850). In some examples, processing circuitry 50 may determine a degree of risk of a fall event based on the one or more parameters of the ECG signal during the second period of time in relation to the generated ECG parameter fall event threshold. For example, based on one or more parameters of the ECG signal during the second period of time and the ECG parameter fall event threshold, processing circuitry 50 may output an indication that a patient has a 90% risk of a fall event within a third period of time. In such an example, processing circuitry 50 may also output a warning, such as an alarm, to warn patient of the severity of the situation. In another example, based on one or more parameters of the ECG signal during the second period of time and the ECG parameter fall event threshold, processing circuitry 50 may output an indication that a patient has a 10% risk of a fall event within a third period of time. In some examples, the third period of time may be after the output of the indication. In some examples, the third period of time may end within one hour after the output of the indication.

For example, if processing circuitry 50 determines an ECG signal during the second period of time includes a pause duration that satisfies the generated personalized pause duration threshold (e.g., an ECG signal parameter fall event threshold), processing circuitry 50 may output an indication the a degree of risk of a fall event within a third period of time based, at least in part, on the duration of pause duration in relation to the generated personalized pause duration threshold.

In some examples, the system 2 or IMD 10 outputting an indication of a degree of risk of a fall event a period of time, such as an hour, may inform a patient of a pending potential catastrophic fall event so the patient may act to reduce the chances of the fall event from occurring and/or reduce the potential harm of the fall event. For example, a patient may sit down, seek medical help, contact an emergency responder, contact a friend/family member, etc. in response to receiving an indication that the patient is at risk of a fall event within a period of time.

In some examples, processing circuitry 50 may utilize machine learning, such as a deep learning algorithm or model (e.g., a neural network or deep belief network), to determine whether one or more parameters of the ECG signal during a period of time indicate a presence of a cardiac event or determine a degree of risk of a fall based on parameters of an ECG signal.

Processing circuitry 50 may be configured to execute an artificial intelligence (AI) engine that operates according to one or more models, such as machine learning models. Machine learning models may include any number of different types of machine learning models, such as neural networks, deep neural networks, convolution neural networks, recurrent neural networks, such as long short term memory networks, dense neural networks, and the like. In some examples, various feature inputs to the AI engine may be fed as direct inputs to different layers in a network and not necessarily prior to the convolution layers. Although described with respect to machine learning models, the techniques described in this disclosure are also applicable to other types of AI models, including rule-based models, finite state machines, and the like. For example, the techniques described in this disclosure are also applicable to Bayesian Belief Networks (BBN) or Bayesian machine learning models (these sometimes referred to as Bayesian Networks or Bayesian frameworks herein), Markov random fields, graphical models, AI models (e.g., Naive Bayes classifiers or deep learning models), and/or other belief networks, such as sigmoid belief networks, deep belief networks (DBNs), etc. In other examples, the disclosed technology may leverage non-Bayesian prediction or probability modeling, such as frequentist inference modeling or other statistical models.

Machine learning may generally enable a computing device to analyze input data and identify an action to be performed responsive to the input data. Each machine learning model may be trained using training data that reflects likely input data. The training data may be labeled or unlabeled (meaning that the correct action to be taken based on a sample of training data is explicitly stated or not explicitly stated, respectively).

The training of the machine learning model may be guided (in that a designer, such as a computer programmer, may direct the training to guide the machine learning model to identify the correct action in view of the input data) or unguided (in that the machine learning model is not guided by a designer to identify the correct action in view of the input data). In some instances, the machine learning model is trained through a combination of labeled and unlabeled training data, a combination of guided and unguided training, or possibly combinations thereof. Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

Processing circuitry 50 may utilize machine learning, such as a deep learning algorithm or model (e.g., a neural network or deep belief network), to determine whether parameters of the ECG signal during a period of time indicate a presence of a cardiac event or determine a degree of risk of a fall event based on one or more parameters of an ECG signal. Processing circuitry 50 may train a deep learning model to represent a relationship of one or more parameters of the ECG signal to a fall event or relationship of one or more parameters of an ECG signal to a cardiac event. For example, processing circuitry 50 may train the deep learning model using ECG signals from other patients. In some examples, processing circuitry 50 may train the deep leaning model by adjusting the weights of a hidden layer of a neural network model to balance the contribution of each input (e.g., characteristics of the cardiac sound features) according to determining one or more parameters of the ECG signal indicate a presence of a cardiac event or determining a degree of risk of a fall event based on one or more parameters of an ECG signal.

Once the deep learning model is trained, processing circuitry 50 may obtain and apply data, such as the extracted cardiac sound features, to the trained deep learning model.

Parameters of patient 4 that may be used as inputs for determining a risk of fall event in addition to parameters of an ECG signal may include heart rate metrics, heart rate variability metrics, patient activity metrics, arrhythmia metrics, pacing therapy metrics, respiration metrics, and/or metrics of congestion, perfusion, or edema, such as may be determined based on measurements of an impedance of the patient.

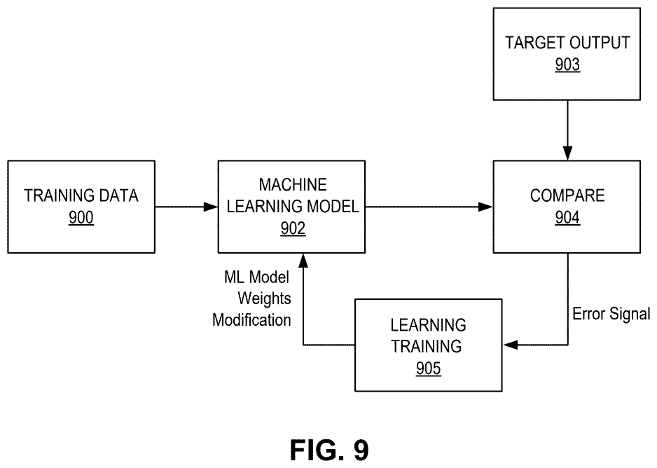

FIG. 9 is an example of a machine learning model 902 and/or machine learning model 1100 being trained using supervised and/or reinforcement learning techniques, such as machine learning model 1100. Machine learning model 902 may correspond to any machine learning model described herein. The machine learning model 902 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, external device 12, server 94, and/or computing device(s) 100 initially trains the machine learning model 902 based on a training set of metrics and corresponding to a HF status. The training set 900 may include a set of feature vectors, where each feature in the feature vector represents a value for a particular metric. One or more of IMD 10, external device 12, server 94, and/or computing device(s) 100 may select a training set comprising a set of training instances, each training instance comprising an association between one or more parameters of the ECG signal and a cardiac event or one or more parameters of the ECG signal degree of risk of a fall event. A prediction or classification by the machine learning model 902 may be compared 904 to the target output 903, and an error signal and/or machine learning model weights modification may sent/applied to the machine learning model 902 based on the comparison to learn/train 905 the machine learning model to modify/update the machine learning model 902. For example, one or more of IMD 10, external device 12, server 94, and/or computing device(s) 100 may, for each training instance in the training set, modify, based on the respective ECG signal parameters, and/or cardiac event status, and/or degree of risk of a fall event of the training instance, the machine learning model 902 to change a score generated by the machine learning model 902 in response to subsequent cardiac heart sound features applied to the machine learning model 902.

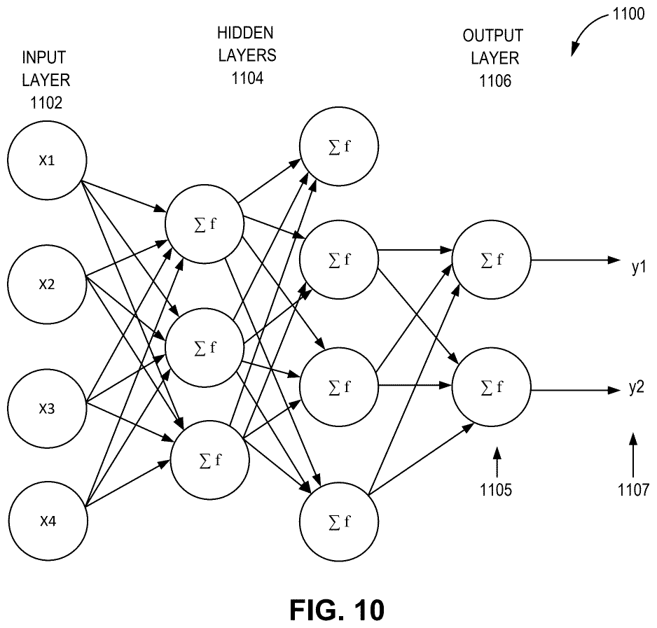

FIG. 10 is a conceptual diagram illustrating an example machine learning model 1100 configured to generate one or more values indicative of a risk of a fall event, based on physiological parameter values, e.g., parameters of an ECG signal. Machine learning model 1100 is an example of a deep learning model, or deep learning algorithm. One or more of IMD 10, external device 12, or server 94 may train, store, and/or utilize machine learning model 1100, but other devices may apply inputs associated with a particular patient to machine learning model 1100 in other examples. Some non-limiting examples of machine learning techniques include Bayesian probability models, Support Vector Machines, K-Nearest Neighbor algorithms, and Multi-layer Perceptron.

As shown in the example of FIG. 10, machine learning model 1100 may include three layers. These three layers include input layer 1102, hidden layer 1104, and output layer 1106. Output layer 1106 comprises the output from the transfer function 1105 of output layer 1106. Input layer 1102 represents each of the input values X1 through X4 provided to machine learning model 1100. The number of inputs may be less than or greater than 4, including much greater than 4, e.g., hundreds or thousands. In some examples, the input values may be any of the of physiological or other patient parameter values described herein.

Each of the input values for each node in the input layer 1102 is provided to each node of hidden layer 1104. In the example of FIG. 10, hidden layers 1104 include two layers, one layer having four nodes and the other layer having three nodes, but fewer or greater number of nodes may be used in other examples. Each input from input layer 1102 is multiplied by a weight and then summed at each node of hidden layers 1104. During training of machine learning model 1100, the weights for each input are adjusted to establish the relationship between input physiological parameter values and one or more output values indicative of a risk level of a health event for the patient. In some examples, one hidden layer may be incorporated into machine learning model 1100, or three or more hidden layers may be incorporated into machine learning model 1100, where each layer includes the same or different number of nodes.

The result of each node within hidden layers 1104 is applied to the transfer function of output layer 1106. The transfer function may be liner or non-linear, depending on the number of layers within machine learning model 1100. Example non-linear transfer functions may be a sigmoid function or a rectifier function. The output 1107 of the transfer function may be a value or values indicative of a presence of a cardiac event or a degree of risk of a fall event or other health event of the patient. By applying the patient parameter data to a machine learning model, such as machine learning model 1100, processing circuitry of system 2 is able to determine a presence of a cardiac event or a degree of risk of a fall event with great accuracy, specificity, and sensitivity.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various aspects of the techniques may enable the following examples.

Example 1: A system includes an implantable medical device includes determine the patient suffered a fall event; in response to the determination that the patient suffered the fall event, determine whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identify the fall event as a cardiac fall event.

Example 2: The system of example 1, wherein the processing circuitry is further configured to: in response to a determination that the one or more parameters of the ECG signal during the period of time indicate the presence of a cardiac event, generate an ECG signal parameter fall event threshold based on the one or more parameters of the ECG signal during the period of time.

Example 3: The system of example 2, wherein the period of time is a first period of time, and the processing circuitry is further configured to: determine whether one or more parameters of an ECG signal during a second period of time satisfies the ECG parameter fall event threshold, the second period of time being after the time the fall event occurs; and in response to a determination that the one or more parameters of the ECG signal during the second period of time satisfies the ECG parameter fall event threshold, output an indication the patient is at risk of a fall event within a third period of time, the third period of time being after the output of the indication.

Example 4: The system of example 2, wherein the period of time is a first period of time, and the processing circuitry is further configured to: determine whether one or more parameters of an ECG signal during a second period of time satisfy the ECG parameter fall event threshold, the second period of time being after the time the fall event occurs; and in response to a determination that the one or more parameters of the ECG signal during the second period of time satisfies the ECG parameter fall event threshold, output an indication of a degree of risk of a fall event by the patient within a third period of time, the third period of time being after the output of the indication.

Example 5: The system of any of examples 3-4, wherein the third period of time ends within one hour after the output of the indication.

Example 6: The system of any of examples 1-5, wherein a duration of the period of time is up to 15 minutes.

Example 7: The system of any of examples 1-6, wherein the implantable medical device further comprises an accelerometer configured to sense an accelerometer signal, and the processing circuitry is further configured to determine the patient suffered the fall event based on the sensed accelerometer signal.

Example 8: The system of any of examples 1-7, wherein the cardiac event is an arrhythmia.

Example 9: The system of any of examples 1-8, wherein the parameters of the ECG signal include at least one of a cardiac pause duration, a duration of arrhythmia, a duration of ventricular tachycardia (VT), a duration of ventricular fibrillation (VF), QRS morphology, T-wave morphology, QT interval, ST elevation, presence of premature ventricular contraction (PVC), morphology of PVC, PVC burden, occurrence of couplet, or occurrence of triplet.

Example 10: An insertable cardiac monitor (ICM) includes a housing configured for subcutaneous implantation within a patient; a plurality of electrodes on the housing, wherein the ICM is configured to sense an electrocardiogram (ECG) signal of the patient via the plurality of electrodes; and processing circuitry configured to: determine the patient suffered a fall event; in response to the determination that the patient suffered the fall event, determine whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identify the fall event as a cardiac fall event.

Example 11: The ICM of example 10, wherein the ICM further comprises an accelerometer within the housing, wherein the ICM is configured to sense an accelerometer signal via the accelerometer, and the processing circuitry is further configured to determine the patient suffered the fall event based on the sensed accelerometer signal.

Example 12: The ICM of any of examples 10-11, wherein the processing circuitry is further configured to: in response to a determination that the one or more parameters of the ECG signal during the period of time indicate the presence of a cardiac event, generate an ECG signal parameter fall event threshold based on the one or more parameters of the ECG signal during the period of time.

Example 13: The ICM of example 12, wherein the period of time is a first period of time, and the processing circuitry is further configured to: determine whether one or more parameters of an ECG signal during a second period of time satisfies the ECG parameter fall event threshold, the second period of time being after the time the fall event occurs; and in response to a determination that the one or more parameters of the ECG signal during the second period of time satisfies the ECG parameter fall event threshold, output an indication the patient is at risk of a fall event within a third period of time, the third period of time being after the output of the indication.

Example 14: The ICM of example 12, wherein the period of time is a first period of time, and the processing circuitry is further configured to: determine whether one or more parameters of an ECG signal during a second period of time satisfies the ECG parameter fall event threshold, the second period of time being after the time the fall event occurs; and in response to a determination that the one or more parameters of the ECG signal during the second period of time satisfies the ECG parameter fall event threshold, output an indication of a degree of risk of a fall event by the patient within a third period of time, the third period of time being after the output of the indication.

Example 15: The ICM of any of examples 13-14, wherein the third period of time ends within one hour after the output of the indication.

Example 16: The ICM of any of examples 10-15, wherein a duration of the period of time is up to 15 minutes.

Example 17: The ICM of any of examples 10-16, wherein the cardiac event is an arrhythmia.

Example 18: The ICM of any of examples 10-17, wherein the one or more parameters of the ECG signal include at least one of a cardiac pause duration, a duration of arrhythmia, a duration of ventricular tachycardia (VT), a duration of ventricular fibrillation (VF), QRS morphology, T-wave morphology, QT interval, ST elevation, or presence of premature ventricular contraction (PVC), morphology of PVC, PVC burden, occurrence of couplet, or occurrence of triplet.

Example 19: A method includes receiving an electrocardiogram (ECG) signal of a patient; receiving a sensed accelerometer signal; determining the patient suffered a fall event based on the sensed accelerometer signal; in response to the determination that the patient suffered the fall event, determining whether one or more parameters of the ECG signal during a period of time indicate a presence of a cardiac event, the period of time being before a time the fall event occurs; and in response to a determination that the one or more parameters of the ECG signal during the period of time indicate the presence of a cardiac event, identifying the fall event as a cardiac fall event.

Example 20: The method of example 19, wherein the method further comprises: in response to a determination that the one or more parameters of the ECG signal during the period of time indicate the presence of a cardiac event, generating an ECG signal parameter fall event threshold based on the one or more parameters of the ECG signal during the period of time.

Example 21: The method of example 20, wherein the period of time is a first period of time, and the method further comprises: determining whether one or more parameters of an ECG signal during a second period of time satisfies the ECG parameter fall event threshold, the second period of time being after the time the fall event occurs; and in response to a determination that the one or more parameters of the ECG signal during the second period of time satisfies the ECG parameter fall event threshold, outputting an indication the patient is at risk of a fall event within a third period of time, the third period of time being after the output of the indication.

Example 22: The method of example 20, wherein the period of time is a first period of time, and the method further comprises: determining whether one or more parameters of an ECG signal during a second period of time satisfies the ECG parameter fall event threshold, the second period of time being after the time the fall event occurs; and in response to a determination that the one or more parameters of the ECG signal during the second period of time satisfies the ECG parameter fall event threshold, outputting an indication of a degree of risk of a fall event by the patient within a third period of time, the third period of time being after the output of the indication.

Example 23: The method of any of examples 21-22, wherein the third period of time ends within one hour after the output of the indication.

Example 24: The method of any of examples 19-23, wherein a duration of the period of time is up to 15 minutes.

Example 25: The method of any of examples 19-24, wherein the cardiac event is an arrhythmia.

Example 26: The method of any of examples 19-25, wherein the one or more parameters of the ECG signal include at least one of a cardiac pause duration, a duration of arrhythmia, a duration of ventricular tachycardia (VT), a duration of ventricular fibrillation (VF), QRS morphology, T-wave morphology, QT interval, ST elevation, or presence of premature ventricular contraction (PVC), morphology of PVC, PVC burden, occurrence of couplet, or occurrence of triplet.

Various examples have been described. These and other examples are within the scope of the following claims.

What is claimed is:

1. A system comprising:
an implantable medical device comprising a plurality of electrodes configured to sense an electrocardiogram (ECG) signal of a patient; and
processing circuitry configured to:
determine the patient suffered a fall event;
in response to the determination that the patient suffered the fall event, determine whether one or more parameters of the ECG signal during a period of time indicate a presence of a cardiac event, the period of time being before a time the fall event occurs; and
in response to a determination that the one or more parameters of the ECG signal during the period of time indicate the presence of a cardiac event, identify the fall event as a cardiac fall event.

2. The system of claim 1, wherein the processing circuitry is further configured to:
in response to a determination that the one or more parameters of the ECG signal during the period of time indicate the presence of a cardiac event, generate an ECG signal parameter fall event threshold based on the one or more parameters of the ECG signal during the period of time.

3. The system of claim 2, wherein the period of time is a first period of time, and the processing circuitry is further configured to:
determine whether one or more parameters of an ECG signal during a second period of time satisfies the ECG parameter fall event threshold, the second period of time being after the time the fall event occurs; and
in response to a determination that the one or more parameters of the ECG signal during the second period of time satisfies the ECG parameter fall event threshold, output an indication the patient is at risk of a fall event within a third period of time, the third period of time being after the output of the indication.

4. The system of claim 2, wherein the period of time is a first period of time, and the processing circuitry is further configured to:
determine whether one or more parameters of an ECG signal during a second period of time satisfy the ECG parameter fall event threshold, the second period of time being after the time the fall event occurs; and
in response to a determination that the one or more parameters of the ECG signal during the second period of time satisfies the ECG parameter fall event threshold, output an indication of a degree of risk of a fall event by the patient within a third period of time, the third period of time being after the output of the indication.

5. The system of claim 3, wherein the third period of time ends within one hour after the output of the indication.

6. The system of claim 1, wherein a duration of the period of time is up to 15 minutes.

7. The system of claim 1, wherein the implantable medical device further comprises an accelerometer configured to sense an accelerometer signal, and the processing circuitry is further configured to determine the patient suffered the fall event based on the sensed accelerometer signal.

8. The system of claim 1, wherein the cardiac event is an arrhythmia.

9. The system claim 1, wherein the parameters of the ECG signal include at least one of a cardiac pause duration, a duration of arrhythmia, a duration of ventricular tachycardia (VT), a duration of ventricular fibrillation (VF), QRS morphology, T-wave morphology, QT interval, ST elevation, presence of premature ventricular contraction (PVC), morphology of PVC, PVC burden, occurrence of couplet, or occurrence of triplet.

10. An insertable cardiac monitor (ICM) comprising:
a housing configured for subcutaneous implantation within a patient; a plurality of electrodes on the housing, wherein the ICM is configured to sense an electrocardiogram (ECG) signal of the patient via the plurality of electrodes; and
processing circuitry configured to:
determine the patient suffered a fall event;
in response to the determination that the patient suffered the fall event, determine whether one or more parameters of the ECG signal during a period of time indicate a presence of a cardiac event, the period of time being before a time the fall event occurs; and
in response to a determination that the one or more parameters of the ECG signal during the period of time indicate the presence of a cardiac event, identify the fall event as a cardiac fall event.

11. The ICM of claim 10, wherein the ICM further comprises an accelerometer within the housing, wherein the ICM is configured to sense an accelerometer signal via the accelerometer, and
the processing circuitry is further configured to determine the patient suffered the fall event based on the sensed accelerometer signal.

12. The ICM of claim 10, wherein the processing circuitry is further configured to:
in response to a determination that the one or more parameters of the ECG signal during the period of time indicate the presence of a cardiac event, generate an ECG signal parameter fall event threshold based on the one or more parameters of the ECG signal during the period of time.

13. The ICM of claim 12, wherein the period of time is a first period of time, and the processing circuitry is further configured to:
determine whether one or more parameters of an ECG signal during a second period of time satisfies the ECG parameter fall event threshold, the second period of time being after the time the fall event occurs; and
in response to a determination that the one or more parameters of the ECG signal during the second period of time satisfies the ECG parameter fall event threshold, output an indication the patient is at risk of a fall event within a third period of time, the third period of time being after the output of the indication.

14. The ICM of claim 12, wherein the period of time is a first period of time, and the processing circuitry is further configured to:
determine whether one or more parameters of an ECG signal during a second period of time satisfies the ECG parameter fall event threshold, the second period of time being after the time the fall event occurs; and
in response to a determination that the one or more parameters of the ECG signal during the second period of time satisfies the ECG parameter fall event threshold, output an indication of a degree of risk of a fall event by the patient within a third period of time, the third period of time being after the output of the indication.

15. The ICM of claim 13, wherein the third period of time ends within one hour after the output of the indication.

16. The ICM of claim 10, wherein a duration of the period of time is up to 15 minutes.

17. The ICM of claim 10, wherein the one or more parameters of the ECG signal include at least one of a cardiac pause duration, a duration of arrhythmia, a duration of ventricular tachycardia (VT), a duration of ventricular fibrillation (VF), QRS morphology, T-wave morphology, QT interval, ST elevation, or presence of premature ventricular contraction (PVC), morphology of PVC, PVC burden, occurrence of couplet, or occurrence of triplet.

18. A method comprising:
receiving an electrocardiogram (ECG) signal of a patient;
receiving a sensed accelerometer signal;
determining the patient suffered a fall event based on the sensed accelerometer signal;
in response to the determination that the patient suffered the fall event, determining whether one or more parameters of the ECG signal during a period of time indicate a presence of a cardiac event, the period of time being before a time the fall event occurs; and
in response to a determination that the one or more parameters of the ECG signal during the period of time indicate the presence of a cardiac event, identifying the fall event as a cardiac fall event.

19. The method of claim 18, wherein the method further comprises:
in response to a determination that the one or more parameters of the ECG signal during the period of time indicate the presence of a cardiac event, generating an ECG signal parameter fall event threshold based on the one or more parameters of the ECG signal during the period of time.

20. The method of claim 19, wherein the period of time is a first period of time, and the method further comprises:
determining whether one or more parameters of an ECG signal during a second period of time satisfies the ECG parameter fall event threshold, the second period of time being after the time the fall event occurs; and
in response to a determination that the one or more parameters of the ECG signal during the second period of time satisfies the ECG parameter fall event threshold, outputting an indication the patient is at risk of a fall event within a third period of time, the third period of time being after the output of the indication.
